(19) United States
(12) Patent Application Publication
(10) Pub. No.: US 2009/0048196 A1
(43) Pub. Date: Feb. 19, 2009

(54) Membrane receptor for retinol binding protein mediates cellular uptake of vitamin A, methods of use and compositions

(75) Inventors: Hui Sun, Culver City, CA (US); Riki Kawaguchi, Tarzana, CA (US); Dean Bok, Los Angeles, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/016,883

(22) Filed: Jan. 18, 2008

Related U.S. Application Data
(60) Provisional application No. 60/885,576, filed on Jan. 18, 2007.

(51) Int. Cl. A61K 31/7105; C12Q 1/02; G01N 33/53; A01K 67/027; C12N 5/06; C12Q 1/68

(52) U.S. Cl. 514/44; 435/29; 435/7.2; 435/6; 435/325; 800/13

(57) ABSTRACT

Provided is a method of diagnosing a condition associated with abnormal uptake of a retinoid comprising determining the amount of STRA6 expressed by a suspect cell and comparing the amount of expressed STRA6 in the suspect cell with the amount of expressed STRA6 in a normal cell. Also provided is a method for treating a condition associated with excessive uptake or insufficient uptake of a retinoid comprising administering to a subject in need thereof an amount of a drug effective for lowering or increasing expression or activity of STRA6 in cells. Also provided is a method of screening for a compound which alters the uptake of a retinoid by a cell comprising exposing the cell to the compound and determining if the expression or activity of STRA6 is altered.

### CROSS-REFERENCE TO RELATED APPLICATIONS

This application claims the benefit of U.S. Provisional Application Ser. No. 60/885,576, filed on Jan. 18, 2007, the disclosure of which is incorporated herein by reference.

### FIELD OF THE INVENTION

The invention relates to uses of the membrane receptor for retinal binding protein.

### BACKGROUND

Currently, the most common therapeutic uses of retinoids are in dermatology and oncology. Retinoids have been used to treat various types of cancer and various skin diseases such as psoriasis and other hyperkeratotic and parakeratotic skin disorders, keratotic genodermatoses, and severe acne and acne-related dermatoses. Given the potent biological effects of retinoids, current retinoid treatment is associated with diverse toxic effects such as teratogenicity, bone toxicity and serum lipid increments. Therefore, there is a need for improved approaches of retinoid therapy.

### SUMMARY

The disclosure provides a method of diagnosing a condition associated with abnormal uptake of a retinoid. The method comprises determining the amount of STRA6 expressed by a suspect cell and comparing the amount of expressed STRA6 in the suspect cell with the amount of expressed STRA6 in a normal cell.

In another aspect, the disclosure is directed to a method of treating a condition associated with excessive uptake of a retinoid. The method comprises administering to a subject in need thereof an amount of a drug effective for lowering expression or activity of STRA6 in cells with excessive retinoid uptake.

In still another aspect, the disclosure is directed to a method of treating a condition associated with insufficient uptake of a retinoid. The method comprises administering to a subject in need thereof an amount of a drug effective for increasing expression or activity of STRA6 in cells with insufficient retinoid uptake.

In yet another aspect, the disclosure is directed to a method of screening for a compound which alters the uptake of a retinoid by a cell. The method comprises exposing the cell to the compound and determining if the expression or activity of STRA6 is altered.

In another aspect, the disclosure is directed to a transfected cell comprising exogenous DNA encoding STRA6. Yet another aspect of the disclosure is directed to a transgenic animal wherein the animal comprises cells transfected with DNA encoding STRA6.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1A-C shows a development strategy and data obtained from the strategy. A. Schematic diagram of the strategy used to identify the RBP receptor. The structure of the amino-reactive and photo-reactive crosslinker is shown on the top and illustrated as red symbols in the diagram. The letter H above the symbol for RBP represents the 6×His tag at the N-terminus. Shaded rectangles represent membrane. B. Binding of AP-RBP to freshly dissected bovine RPE cells (upper picture). AP reaction product is deep purple. Presence of 10-fold excess RBP abolished the labeling (lower picture). The hexagonal shape of an RPE cell is outlined in red dotted lines in each picture. Bar, 10 μm. C. Immunoblot analysis using an anti-RBP antibody. Molecular weight marker is shown on the left (in kD). The His-RBP monomer band represents His-RBP that was bound to RPE membrane but was not crosslinked to the receptor. Untagged RBP competition in lane 4 prevented His-RBP from binding to RPE membrane and therefore lowered signals for both the complex and the monomer band.

FIG. 2A-H shows RBP binding and vitamin A uptake activity of STRA6. A. Binding of RBP to live COS-1 cells transfected with STRA6 (upper picture) but not to untransfected cells (lower picture). The AP reaction product is deep purple. Bar, 20 μm. B. Concentration dependence of the binding of AP-RBP to live COS-1 cells transfected with STRA6. Controls are LRAT-transfected cells, untransfected cells and STRA6-transfected cells with 50× unlabeled RBP during binding. The plots for these 3 controls are superimposed on this graph. C. Comparison of vitamin A uptake activities from ³H-retinol/RBP. U, S, L, and S/L denote untransfected cells, cells transfected with STRA6, LRAT and both, respectively. RBP, 100× unlabeled holo-RBP. The activity of S/L cells is defined as 100%. D-H. HPLC assay of vitamin A uptake for COS-1 cells transfected with STRA6 and LRAT, LRAT alone, and untransfected cells. D. HPLC profile of hexane extracts of COS-1 cells after vitamin A uptake from holo-RBP. An arrowhead indicates the peak representing retinyl ester. A peak representing hexane-soluble peptides (asterisk) serves as an internal control. E. Overlay of the retinyl ester peaks in D. F. Overlay of absorption spectra of the retinyl ester peaks in D. G. Overlay of the retinyl ester peaks from similar HPLC runs as shown in D but using 25% human serum as the source of holo-RBP. H. Overlay of absorption spectra of the peaks in G.

FIG. 3A-E shows STRA6 RNAi in native cell types and STRA6 mutagenesis. A. Retinoic acid (RA) treatment of WiDr cells increases the expression of STRA6 and vitamin A uptake activity of WiDr cells, while STRA6 RNAi treatment suppresses STRA6 expression and vitamin A uptake activity. NC is negative control RNAi oligo. S-1, S-2 and S-3 are RNAi oligos for human STRA6. B. STRA6 RNAi treatment suppresses STRA6 expression and vitamin A uptake activity of primary bovine RPE culture. NC is negative control RNAi oligo. S-4, S-5 and S-6 are RNAi oligos for bovine STRA6. C. Comparison of vitamin A uptake activities of COS-1 cells transfected with bovine STRA6 wild-type (WT) or mutants Q310H/L315P (M1), L256H/Y336H (M2), and L297R (M3). Activity of cells transfected with WT STRA6 is defined as 100%. D. Comparison of AP-RBP binding activities of COS-1 cells transfected with bovine STRA6 WT, mutant M1, M2 or M3. Activity of cells transfected with WT STRA6 is defined as 100%. E. Surface expression of wild-type or mutant STRA6 as assayed by immunostaining of transfected live COS-1 cells.

FIG. 4A-F shows characterization of STRA6-mediated vitamin A uptake from holo-RBP. A. STRA6 mediates ³H-retinol uptake from ³H-retinol bound to RBP but not ³H-retinol bound to BSA or β-lactoglobulin. “Transfected” indicates transfection with STRA6 and LRAT. Retinol uptake activity of transfected cells from holo-RBP is defined as 100%. B. STRA6-mediated vitamin A uptake is saturable with regard to retinol-RBP concentration. The highest retinol uptake activity at 60 min is defined as 100%. C. MI (a mixture of metabolic inhibitors 2-deoxyglucose and sodium azide) effectively inhibits HRP endocytosis of COS-1 cells (left graph) but does not inhibit the vitamin A uptake activity of COS-1 cells transfected with STRA6 and LRAT (S/L) (right graph). L, LRAT transfected cells. The activity of S/L cells without MI is defined as 100%. D. MI effectively inhibits HRP endocytosis by WiDr cells (left graph) but does not inhibit vitamin A uptake activity of WiDr cells (right graph). The activity of WiDr cells treated with 40 μM retinoic acid but without MI is defined as 100%. E. Vitamin A uptake activity of membrane fractions of HEK293 cells. U, S, L, and S/L denote membranes from untransfected cells, cells transfected with STRA6, LRAT and both, respectively. R, 100× holo-RBP. The activity of S/L cells is defined as 100%. F. RBP remains outside the cell after STRA6-mediated vitamin A uptake from holo-RBP for 8 hours. Absorption spectra of RBP that remains in the supernatant of untransfected cells (left) and cells transfected with STRA6 and LRAT (right) are shown.

FIG. 5A-H shows immunolocalization of STRA6. Green signal is STRA6. Red signal is peropsin, an apical RPE marker (A) or GSL I-isolectin B4, an endothelial cell marker (B, D, F, G and H). Blue signal is nuclear stain DAPI. Bars represent 10 μm in A, B, and C and 40 μm in D, E, F, G and H. CH, choroid. OS, photoreceptor outer segments. ONL, outer nuclear layer. INL, inner nuclear layer. A & B. STRA6 is localized to the basolateral membrane of the RPE. Arrowheads indicate signals on the lateral membrane of RPE cells. C. A tangential section of the RPE layer. D.& E. STRA6 signals are most enriched in the RPE layer of the eye, but are also present in the blood vessels in retina. E is an overexposed picture of D to show the weaker signals in retina blood vessels. F. Localization of STRA6 in hippocampus. Examples of large blood vessels positive for STRA6 (large arrowhead) and negative for STRA6 (small arrowhead). The STRA6 negative vessel is surrounded by STRA6 positive astrocyte perivascular endfeet. G. Localization of STRA6 in placenta. H. STRA6 is highly expressed in a subset of cells in the spleen.

FIG. 6A-C shows MS spectral analysis of tryptic peptides from the RBP receptor/RBP complex and topographical and sequence characterization of STRA6. A. MS/MS spectrum of a tryptic peptide from the RBP receptor/RBP complex. The peptide LGEEEEGIQLLQTK (SEQ ID NO:13) is identified as a fragment from bovine STRA6 with a MASCOT score of 61. No other peptide was identified due to the extreme hydrophobicity of STRA6. B. Schematic diagram of transmembrane topology of STRA6. Two independent transmembrane domain prediction softwares, TMPRED and SOSUI, predicted that STRA6 has 11 transmembrane domains. Arrowhead indicates the position where BBS is inserted for cell surface expression study. Asterisks indicate the positions of the missense mutations in this study. M1. M2. M3. C. Alignment of human STRA6 isoform 1 (H1) (SEQ ID NO:14), isoform 2 (H2) (SEQ ID NO:15), bovine STRA6 (B) (SEQ ID NO:16), and mouse STRA6 (M) (SEQ ID NO:17) using Vector NTI's AlignX program. Identical residues are highlighted in yellow.

FIG. 7 shows characterization of vitamin A and retinol uptake by cells cotransfected with STRA6 and LRAT. A. STRA6, but not LRAT activity is the rate limiting factor in vitamin A uptake for cells cotransfected with STRA6 and LRAT. COS-1 cells were cotransfected with the same amount of total DNA but varying STRA6:LRAT ratio. STRA6:LRAT=1:9 (closed diamond), 1:1 (open square), 3:2 (closed circle), 9:1 (open triangle). Vitamin A uptake activity is correlated with increasing STRA6 but not increasing LRAT. The highest retinol uptake activity is defined as 100%. B and C. STRA6-mediated retinol uptake is saturable with regard to retinol-RBP concentration. B. HPLC based assay for retinol uptake. This assay is based on cells cotransfected with STRA6 and LRAT at a 1:1 ratio. Cells were incubated with holo-RBP for 60 min. A peak representing hexane soluble peptides (asterisk) serves as an internal control. Retinyl ester peak is indicated by a number, which represents the concentration of retinol-RBP (in nM) in which the cells were incubated. C. Overlay of absorption spectra of retinyl ester peaks in B. The concentrations of retinol-RBP in which the cells were incubated is indicated for each spectrum. The spectra for the highest concentrations of retinol/RBP overlap each other.

### DETAILED DESCRIPTION

As used herein and in the appended claims, the singular forms “a,” “and,” and “the” include plural referents unless the context clearly dictates otherwise. Thus, for example, reference to “a receptor” includes a plurality of such receptors and reference to “the agent” includes reference to one or more agents, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

As used herein “retinoids” refers to vitamin A and any of its derivative, including retinol, retinyl ester, retinal and retinoic acid.

Vitamin A and its derivatives are essential for vision and many other biological processes since they are involved in the proliferation and differentiation of many cell types throughout life. The majority of dietary vitamin A is stored in the liver. The principal physiological carrier of vitamin A (retinol) in the blood for delivery to other organs is retinol binding protein (RBP). RBP is responsible for a well-regulated transport system that provides an evolutionary advantage by helping vertebrates to adapt to fluctuations in vitamin A levels in natural environments. RBP also functions as a signal in insulin resistance. Loss of RBP makes mice extremely sensitive to vitamin A deficiency because the hepatic vitamin A store can no longer be mobilized. Even with a nutritionally complete diet, RBP knockout mice have dramatically lower serum vitamin A levels, similar to the levels in the later stages of vitamin A deficiency in humans. Given the role of vitamin A in immune regulation and the susceptibility of vitamin A-deficient children to infection before visual symptoms, it is likely that the immune system is compromised due to the loss of RBP function even under vitamin A sufficient conditions. Indeed, the circulating immunoglobulin level in RBP knockout mice is half of that in wild-type mice even under vitamin A sufficiency. In humans, loss of RBP function causes a dark adaptation defect and progressive atrophy of the retinal pigment epithelium (RPE) at young ages. Under conditions of vitamin A deficiency, at which wild-type mice behave normally, RBP knockout mice have severe developmental defects in embryos and rapid vision loss in adults after merely a week of vitamin A deficiency. In addition, these mice rapidly develop testicular defects.

It was first shown in the 1970s that there exists a specific cell surface receptor for retinol binding protein on the retinal pigment epithelium (RPE) and intestinal epithelial cells. During the past 3 decades, evidence has also accumulated for the existence of the RBP receptor on other tissue or cell types including the placenta, choroid plexus, testis and macrophages. The cell surface RBP receptor not only specifically binds to RBP, but also mediates vitamin A uptake from vitamin A-loaded RBP (holo-RBP). The disclosure identifies the RBP receptor as STRA6, a widely expressed multitransmembrane domain protein. STRA6 met all three criteria expected of the RBP receptor. First, it conferred RBP binding to transfected cells. Second, it mediated cellular uptake of vitamin A. Third, it was localized to the cellular locations expected of the RBP receptor in native tissues.

The existence of an RBP receptor is supported by a large body of evidence. The absence of vitamin A from abundant erythrocytes and serum albumin, which can bind vitamin A, argue against simple diffusion of retinol from the RBP to the cell membrane as the mechanism of vitamin A delivery by RBP. The fact that free vitamin A can diffuse through membranes is not a good argument against the existence of an RBP receptor. First, virtually all vitamin A in blood is bound to the soluble RBP/TTR complex, which cannot freely pass through cell membranes. Second, there are known examples of molecules that can diffuse through membranes but still depend on membrane transporters to facilitate their transport. For example, prostaglandin can pass the membrane by simple diffusion, but prostaglandin transporter greatly facilitates its transport across membranes. In addition, there is strong experimental evidence supporting the existence of an RBP receptor that mediates cellular vitamin A uptake. The disclosure has identified STRA6 as the RBP receptor. The RBP-STRA6 system represents a small molecule delivery mechanism that involves an extracellular carrier protein but does not depend on endocytosis. Given the potent biological effects (including toxicity) of vitamin A and its derivatives, a controlled release of vitamin A into cells from holo-RBP through STRA6 has an evolutionary advantage over nonspecific diffusion of vitamin A. This mechanism makes it possible to achieve high efficiency and specificity for vitamin A delivery to organs distant from the liver such as the eye, brain, placenta and testis.

STRA6's localization in adult brain is consistent with vitamin A's roles in regulating adult brain activities, such as synaptic plasticity and cortical synchrony during sleep. STRA6 expression in the placenta and testis is consistent with the role of RBP in delivering vitamin A to these tissues. STRA6 expression in the spleen and thymus is consistent with a role for vitamin A in immune regulation and in prevention against infectious disease. According to NCBI's EST analysis, STRA6 is also expressed in human skin and lung, both known to depend on vitamin A for proper function. Undifferentiated human skin keratinocytes were found to have the highest RBP binding activity of any cell or tissue types tested. The absence or very low expression of STRA6 in the liver makes physiological sense. Liver is the major site of production of vitamin A-loaded RBP to deliver vitamin A to peripheral organs. If STRA6 is highly expressed in the liver and absorbs vitamin A from holo-RBP produced by liver itself, a “short circuit” would be created.

STRA6 was originally found to be a retinoic acid-stimulated gene in cancer cell lines. However, it is possible that there are certain non-cancer cell types that respond to retinoic acid to stimulate STRA6 expression. For example, vitamin A combined with retinoic acid increases retinol uptake in the lung in a synergistic manner, consistent with the ability of retinoic acid to stimulate STRA6 expression. One study found that STRA6 was overexpressed up to 172 fold in 14 out of 14 human colorectal tumors relative to the normal colon tissue. Thus, the RBP-STRA6 system not only functions as a physiological mechanism of vitamin A uptake but also potentially participates in pathological processes such as insulin resistance and cancer.

RBP/STRA6 is a natural physiological system for vitamin A delivery to many organs. Increasing retinoid level by stimulating STRA6 activity using pharmacology or molecular biology methods can avoid the toxic effects of systemic administration of retinoids. Upregulating STRA6 activity will only increase uptake through a physiological system. Instead of exposing all cells to retinoids, only cell types that naturally take up vitamin A through STRA6 will take up vitamin A for storage or further delivery. When decreasing the retinoid level is desired in treating certain disease, cell-specific or tissue-specific suppression of STRA6 activity is better than systemic lowering of vitamin A/RBP level in the blood. Given the diverse function of vitamin A in many organs, long term systemic lowering of the vitamin A level is expected to have undesirable side effects.

The present discovery makes it possible to modulate vitamin A level in a target organ as a way to treat disease or alleviate symptoms without using retinoids, which are associated with diverse side effects. After a screen for specific activators and blockers of this receptor, animal studies can be carried out to determine if potential drugs modulate tissue vitamin A uptake. Blocker and activator compounds can be used to treat human disease or alleviate disease symptoms. Gene therapy methods which specifically target STRA6 to increase or decrease its activity may also be used.

Vitamin A is essential for human survival because its derivatives (retinoids) participate in diverse physiological functions such as embryonic growth and development, vision, neuronal signaling, spermatogenesis, and the maintenance of immune competence and epithelium integrity. Imbalance in vitamin A homeostasis can lead to a wide range of human diseases such as blindness, birth defects, neurological disorders and susceptibility to infectious disease. Therefore, modulating tissue vitamin A level by targeting STRA6 can be used to treat or alleviate the symptoms of visual disorders, neurological disorders, cancer, diabetes, skin diseases, immune disorders, and lung diseases. Visual disorders can include, for example, diabetic retinopathy and age-related macular degeneration (both dry and wet forms), and can include treatment of retinal pigmented epithelial cells.

The methods of the disclosure can be used in any mammalian species, including human, monkey, cow, sheep, pig, goat, horse, mouse, rat, dog, cat, rabbit, guinea pig, hamster and horse. Humans are preferred.

In one aspect, the disclosure is directed to a method of diagnosing a condition associated with abnormal uptake of a retinoid. The method comprises determining the amount of STRA6 expressed by a suspect cell and comparing the amount of expressed STRA6 in the suspect cell with the amount of expressed STRA6 in a normal cell. A suspect cell is one from an individual suspected of having a disease or disorder associated with abnormal uptake of a retinoid. A normal cell is one from an individual in good health. Conditions associated with abnormal uptake of a retinoid include conditions in which an insufficient amount of retinoid is taken up by the suspect cell, and conditions in which an excessive amount of retinoid is taken up by the suspect cell.

Any appropriate method may be used to determine the amount of expressed STRA6 in the cells. For example, expressed STRA6 may be determined using an antibody which specifically binds STRA6, including a polyclonal or monoclonal antibody. As another example, expressed STRA6 may be determined using PCR with primers specific for STRA6. Antibodies, probes and primers can be developed using methods known to those of skill in the art based upon the polypeptide and nucleic acid sequences disclosed herein, as well as any naturally-occurring variant nucleic acid and polypeptide sequences of STRA6 and STRA6 sequences such as those accessible through various databases (see, e.g., the following accession numbers available through NCBI the disclosures of which are incorporated herein by reference; AAC16016 (gi|3126975|gb|AAC16016.1|[3126975]); AAQ89447 (gi|37183295|gb|AAQ89447.1|[37183295]); AAQ89108 (gi|37182615|gb|AAQ89108.1|[37182615]); AAI42343 (gi|148745510|gb|AAI42343.1|[148745510]); AAH75657 (gi|49523348|gb|AAH75657.1|[49523348]); AAH15881 (gi|33871375|gb|AAH15881.1|[33871375]); AAH76188 (gi|49902946|gb|AAH76188.1|[49902946])).

Another aspect of the disclosure is directed to a method of treating a condition associated with excessive uptake of a retinoid. The method comprises administering to a subject in need thereof an amount of a drug effective for lowering expression or activity of STRA6 in cells with excessive retinoid uptake. Such a drug may include an appropriate small molecule formulation, wherein the small molecule results in reducing or blocking the activity of STRA6. As another example, the drug may be an siRNA which reduces expression of STRA6, the siRNA being introduced into cells with excessive retinoid uptake. Another alternative is a drug which is an antibody, such as a polyclonal or monoclonal antibody, which specifically binds to and inactivates STRA6.

The siRNAs for use in the disclosure are designed according to standard method in the field of RNA interference. Introduction of siRNAs into cells may be by transfection with expression vectors, by transfection with synthetic dsRNA, by direct injection into cells, or by any other appropriate methods.

The expression vectors which can be used to deliver siRNA according to the invention include retroviral, adenoviral and lentiviral vectors. The expression vector includes a sequence which codes for a portion of a target gene or any other sequence whether specific for a particular gene or a nonsense sequence. The gene sequence is designed such that, upon transcription in the transfected host, the RNA sequence forms a hairpin structure due to the presence of self-complementary bases. Processing within the cell removes the loop resulting in formation of a siRNA duplex. The double stranded RNA sequence should be less than 30 nucleotide bases; preferably the dsRNA sequence is 19-25 bases in length; more preferably the dsRNA sequence is 20 nucleotides in length.

The expression vectors may include one or more promoter regions to enhance synthesis of the target gene sequence. Promoters which can be used include CMV promoter, SV40 promoter, promoter of mouse U6 gene, and promoter of human H1 gene.

One or more selection markers may be included to facilitate transfection with the expression vector. The selection marker may be included within the expression vector, or may be introduced on a separate genetic element. For example, the bacterial hygromycin B phosphotransferase gene may be used as a selection marker, with cells being grown in the presence of hygromycin to select for those cells transfected with the aforementioned gene.

Synthetic dsRNA may also be introduced into cells to provide gene silencing by siRNA. The synthetic dsRNAs are less than 30 base pairs in length. Preferably the synthetic dsRNAs are 19-25 base pairs in length. More preferably the dsRNAs are 19, 20 or 21 base pairs in length, optionally with 2-nucleotide 3′ overhangs. The 3′ overhangs are preferably TT residues.

Synthetic dsRNAs can be introduced into cells by injection, by complexing with agents such as cationic lipids, by use of a gene gun, or by any other appropriate methods.

In another aspect, the disclosure relates to a method of treating a condition associated with insufficient uptake of a retinoid. The method comprises administering to a subject in need thereof an amount of a drug effective for increasing expression or activity of STRA6 in cells with insufficient retinoid uptake. Such a drug may include an appropriate small molecule formulation, wherein the small molecule results in enhancing the activity of STRA6. As another example, the drug may be an exogenous nucleic encoding STRA6 which is transfected into cells with insufficient retinoid uptake, thereby resulting in increased expression of STRA6. Any appropriate method for transfecting a cell with exogenous DNA may be used. Other aspects of the disclosure include a transfected cell, the cell comprising exogenous DNA encoding STRA6; or a transgenic animal, the animal comprising cells transfected with DNA encoding STRA6.

Another aspect of the disclosure is directed to a method of screening for a compound which alters the uptake of a retinoid by a cell. The method comprises exposing the cell to the compound and determining if the expression or activity of STRA6 is altered. The alteration may be either an increase in expression or activity of STRA6, or a decrease in expression or activity of STRA6. Additionally, the method may be performed in vitro, such as in a cell culture, or in vivo, such as in an animal.

### EXAMPLES

Preparation of RPE membranes: Fresh bovine eyes were purchased from a local slaughterhouse. Approximately 40 eyes are routinely processed. RPE cells were dissected from bovine eyes under a red safety light and kept on ice in PBS until all dissections were complete. RPE cells were then centrifuged at 2,000 g for 10 min. The supernatant was discarded and the pellet was stored at −80° C. until use. The RPE cell pellet was suspended in 8.6% sucrose in PBS and protease inhibitors and broken up by polytron for 30 sec on ice. The RPE extracts were layered onto 40% sucrose in PBS and centrifuged at 25,000 g for 30 min. The membrane fraction at the interphase was collected, diluted in PBS and further centrifuged at 25,000 g for 30 min. The membrane pellet was then resuspended in PBS by needle passage and brief sonication.

RBP binding to RPE membrane and UV crosslinking: Earlier studies suggest that crosslinking may be an effective method to generate a complex between RBP and its receptor. Of many crosslinkers tested, the bifunctional crosslinker SANPAH (Pierce), which has both an amine reactive group and a photoreactive group, is effective in creating a stable RBP-receptor complex. The amine-reactive group on the crosslinker was first covalently linked to the purified His-RBP. The His-RBP and crosslinker conjugate was then added to RPE membranes to allow binding. UV irradiation stabilizes the interaction between RBP and its receptor by crosslinking. Twenty five micrograms of purified His-RBP protein in 100 μl of PBS was conjugated with 1 μl of 10 mg/ml of SANPAH dissolved in DMSO for 1 hour on ice. The reaction was stopped by adding glycine to 40 mM. Binding of RBP to RPE membranes was carried out in PBS with 5 mg/ml BSA, 0.5 mM CaCl₂ and 1.7 μM all-trans retinol for 10-20 min at room temperature with gentle shaking. Ten-fold untagged RBP was added for the competition experiments. The reaction volume was typically between 500 and 750 μl. To crosslink RBP to its receptor, long wavelength UV light was applied to each sample at a 5 cm distance for 20 min. The reaction was then diluted with PBS, and centrifuged at 25,000 g for 30 min. The membrane pellet was washed twice with PBS and stored at −20° C.

Purification of the RBP/receptor complex: The covalent RBP-receptor complex was solubilized and purified through the His tag of RBP. Membrane pellets after photocrosslinking were solubilized in 1% dodecylmaltoside and 1 mM CaCl₂ with PBS. The solubilized membrane proteins were diluted by 10 fold with 1% CHAPS and 1 mM CaCl₂ in PBS and bound to Ni-NTA resin (Qiagen) in the presence of 10 mM imidazole for at least 12 h at 4° C. The resin was washed 3 times with 0.1% dodecyl-maltoside, 0.9% CHAPS, 1 mM CaCl₂ and 15 mM imidazole in PBS. The His tag-nickel system permits stringent washing with high salt and urea, which can dissociate non-specifically bound protein without causing membrane protein aggregation. Nonspecifically bound proteins were first washed with 500 mM NaCl, 1% Triton X-100 and 1% NP-40 and then with 8M urea, 1% Triton X-100 and 1% NP-40. Finally, the crosslinked RBP/receptor complex was eluted with PBS containing 250 mM imidazole, 1% Triton X-100 and 1% NP-40. Eluted materials were mixed with SDS-loading buffer and loaded on an SDS-PAGE without boiling for Western blot analysis. An anti-RBP antibody (Accurate Chemical) was used to detect the RBP/receptor complex. For total protein staining, eluted material corresponding to starting materials from 10 cow eyes was resolved in one lane of an SDS-PAGE gel for staining with SYPRO Ruby protein stain (Molecular Probes). The protein band that corresponded to the position of the RBP receptor and was absent in the negative controls was excised for further analysis.

In-gel digest of the RBP/receptor complex: The excised protein gel band was destained twice in 50 mM ammonium bicarbonate in 50% acetonitrile at 37° C. for 10 min. The gel slice was dehydrated by incubating in acetonitrile for 5 min. Protein in the gel slice was then reduced in 10 mM DTT and alkylated in 55 mM iodoacetoamide and digested with trypsin overnight at 37° C. After digestion, peptides were extracted in 1% formic acid and 2% acetonitrile followed by 0.5% formic acid and 50% acetonitrile. Peptide samples were concentrated by Speedvac before mass spectrometry (MS) analysis.

μLC-MS/MS (micro-liquid chromatography with tandem mass spectrometry) and MS/MS Ion Search: Peptide samples were analyzed by μLC-MS/MS with data-dependent acquisition (Q STAR XL, Applied Biosystems) after being dissolved in 10 μL 0.1% formic acid, 5% acetonitrile (v/v). A reverse-phase column (200 μm×10 cm; PLRP/S 5 um, 300 Å; Michrom Biosciences) was equilibrated for 20 min at 2 μL/min with 95% A, 5% B (A, 0.1% formic acid, 5% acetonitrile in water; B, 0.1% formic acid in acetonitrile) prior to sample injection (5 μL). A compound linear gradient was initiated 3 min after sample injection ramping to 80% A, 20% B at 8 min; 65% A, 35% B at 13 min; 25% A, 75% B at 23 min; 90% A, 10% B at 23.1 min. The column eluent was directed to a stainless steel nano-electrospray emitter (ES301; Proxeon, Odense, Denmark) at 4.4 kV for ionization without nebulizer gas. The mass spectrometer was operated in “IDA” mode (information dependent acquisition) with a survey scan (400-1500 m/z), and data-dependent MS/MS on the two most abundant ions, with exclusion after two MSMS experiments. Collision induced dissociation data produced from Q-STAR were analyzed and compiled by Analyst QS software (Applied Biosystems). An MS/MS ion search was conducted on a local MASCOT server against the NCBI NR database using “other mammalian” for taxonomy. Other criteria for the search included tryptic peptide mass tolerance <±1.0 Da, fragment mass tolerance <±0.3 Da, cysteine carbamidomethylation as fixed modification and methionine oxidation as potential modification.

Production, vitamin A loading, and purification of holo-RBP: A published protocol for producing, refolding and purifying vitamin A-loaded RBP was used. Briefly, His-RBP (N-terminal 6×His tag) was expressed in BL-21 cells using a standard protocol. The bacterial cell pellet was frozen and thawed twice, and sonicated. Insoluble material was pelleted and solubilized in 7.5 M guanidine hydrochloride. Buffer (25 mM Tris, pH 9.0) was added to dilute the guanidine hydrochloride concentration to 5.0 M. Proteins were further solubilized overnight in the presence of 10 mM DTT. RBP refolding was started by dropwise addition of 4 volumes of a mixture containing 25 mM Tris (pH 9.0), 0.3 mM cystine, 3.0 mM cysteine, 1 mM EDTA, and 1 mM retinol at 4° C. The refolding reaction was carried out for 5 hours at 4° C. by vigorous mixing. The refolding solution was centrifuged and precipitates were removed. The solution was then dialyzed against PBS at 4° C. overnight. His-RBP was purified on an Ni-NTA column and eluted with 100 mM imidazole. Eluted proteins were concentrated and holo-RBP was purified by HPLC using a weak anion exchange column AX300 (Eprogen, Darien, Ill.) on an Agilent 1100 series liquid chromatography system with a photo-diode array detector. Proteins were separated by a NaCl step gradient (120 mM for 10 min, 220 mM for 10 min, and 1000 mM for 15 min) including 25 mM Tris (8.0) at 1 ml/min. Protein elution was monitored at 280 and 330 nm. Holo-RBP was recovered from the 220 mM NaCl fractions.

Cell transfections: COS-1 cells and HEK293 cells were transfected with Fugene6 transfection reagents (Roche) according to the manufacturer's protocol. One well in a 6-well, 12-well or 24-well cell culture plate was transfected with 2 μg, 1 μg or 0.5 μg of DNA, respectively. To compare cells transfected with one plasmid (e.g., LRAT) with cells transfected with two plasmids (e.g., STRA6 and LRAT), a plasmid expressing EGFP was cotransfected with the one plasmid so that every well would get an identical amount of total DNA (e.g., 1 μg of EGFP and LRAT plasmids vs 1 μg of STRA6 and LRAT plasmids). Equalization of the total amount of DNA between wells eliminates the significant effect of DNA quantity on transfection efficiency. All transfections involving STRA6 used bovine STRA6. All assays including RBP binding and vitamin A uptake assays were performed 24 h after transfection.

HPLC-based assay for vitamin A uptake from holo-RBP: Transfected or untransfected cells grown in 6-well cell culture plates were washed with PBS once and incubated with HPLC-purified holo-RBP or pooled human sera (Innovative Research) diluted in serum free medium (SFM) for 4 h or 6 h. Cells were then washed with PBS twice, harvested in PBS containing 5 mM EDTA and pelleted by centrifugation at 1,000 g for 3 min. The cell pellet was resuspended in cold PBS and briefly sonicated. One volume of 1 mM BHT (2,6-Di-tert-butyl-4-methylphenol) in ethanol was added. Retinyl esters were then extracted 3 times with 2 volumes of hexane. The hexane extracts were combined and subjected to HPLC analysis. Samples were analyzed on an Agilent 1100 series liquid chromatography system with a photo-diode array detector. Spectral data were collected throughout the HPLC run. Retinyl esters were separated through an Agilent ZORBAX Rx-SIL column (4.6×250 mm, 5 um). The mobile phase was 1% dioxane and 99% hexane with a flow rate of 2 ml/min. Retinyl esters were detected at an absorbance of 325.4 nm. The identity of retinyl esters was determined by the elution time and spectrum of the corresponding chromatogram peak. Levels of retinyl palmitate were determined from a standard curve with known retinyl palmitate and sample peak areas. The relatively lower signals for untransfected cells in the HPLC-based assay compared with the ³H-retinol-based assay is likely due to the large quantity of retinol/RBP used in the HPLC-based assay, which minimized the effect of non-specific binding of retinol/RBP to plastic.

Production of ³H-retinol loaded RBP and retinol uptake assay based on ³H-retinol/RBP: Apo-RBP was prepared from holo-RBP as described (3). Radioactive retinol (15-³H(N)-retinol) was purchased from American Radiolabeled Chemicals Inc. Ten micrograms apo-His-RBP was incubated with 150 pmol retinol (3 μCi) at 24° C. overnight. Radiolabeled holo-His-RBP was bound to Ni-NTA resin and the resin was washed extensively with 10 mM imidazole in PBS. Radiolabeled His-RBP was eluted with 100 mM imidazole in PBS. For the vitamin A uptake assay, transfected or untransfected cells grown on 12-well or 24-well cell culture plates were washed once with PBS and incubated with the ³H-retinol/RBP diluted in SFM for various lengths of time. The reactions were stopped by washing the cells with PBS and solubilizing cells in 1% Triton X-100 in PBS. Radioactivity was measured with a scintillation counter.

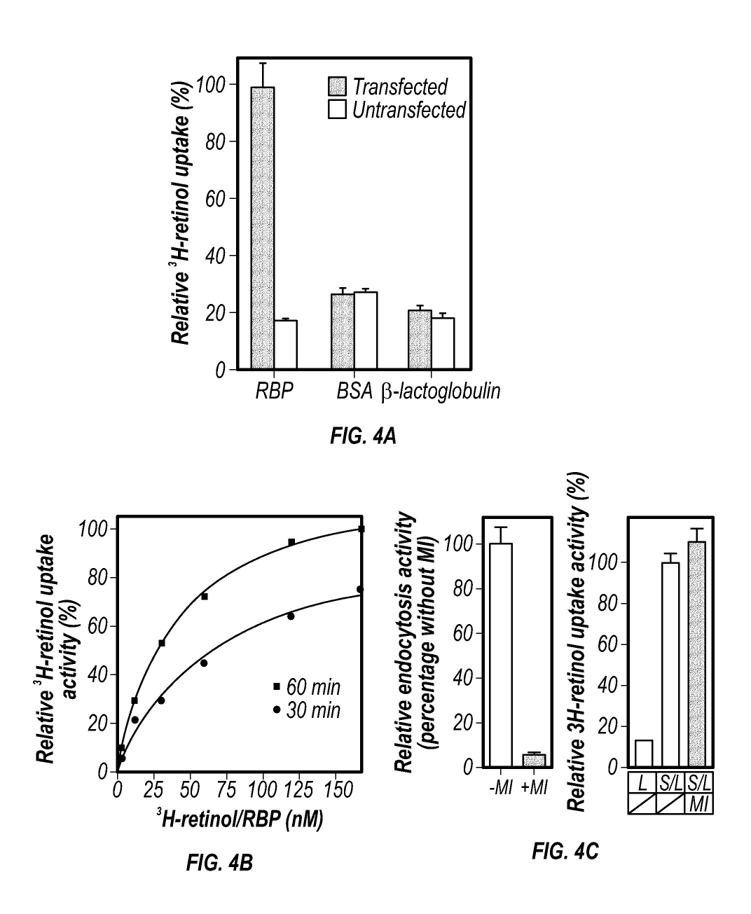

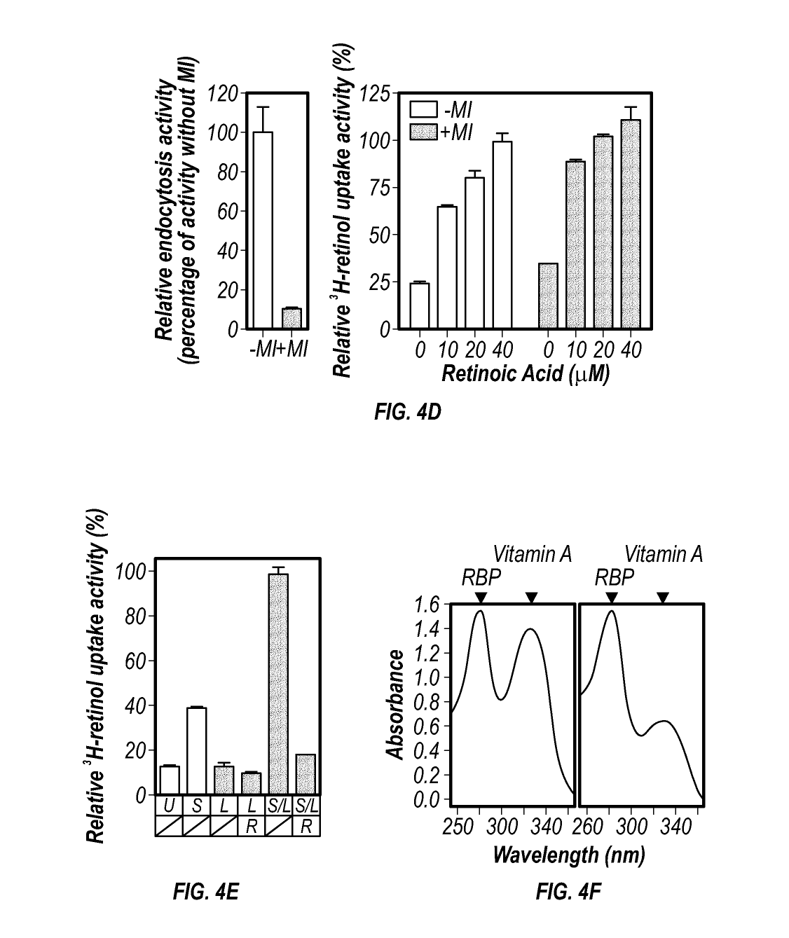

Effects of metabolic inhibitors on vitamin A uptake and endocytosis: To test the effect of metabolic inhibitors, cells were pretreated with metabolic inhibitors in serum free medium (50 mM 2-deoxyglucose and 5 mM sodium azide) at 37° C. for 30 min before incubation with ³H-retinol/RBP for 45 min at 37° C. in the continued presence of the metabolic inhibitors. The slight increase in activity for MI-treated cells as shown in FIGS. 4C and 4D is reproducible. Endocytosis assay based on horseradish peroxidase (HRP) was performed similarly as described. Cells (80-100% confluence) were washed once with PBS and then incubated in SFM with or without metabolic inhibitors for 30 min at 37° C. HRP (1 mg/ml) diluted in SFM containing 0.2% BSA with or without metabolic inhibitors was added to cells preincubated with or without metabolic inhibitors, respectively. After incubation at 37° C. for 1 hour, the cells were washed four times with SFM, harvested in 1×PBS/5 mM EDTA, washed one more time with PBS and lysed in 1% Triton X-100 in PBS with protease inhibitors. Cell lysates were assayed for HRP activity using the 1-STEP Turbo TMB-ELISA reagent (Pierce).

Comparison of retinol uptake from ³H-retinol bound to RBP, BSA and β-lactoglobulin. To produce ³H-retinol-bound BSA and β-lactoglobulin, 1 mg/ml BSA and β-lactoglobulin were incubated with 0.5 nM ³H-retinol (30 μCi/ml) for 1 h at room temperature. Unbound retinol was removed by passing twice through desalting columns (Pierce). Equal amounts of radioactivity (21,000 CPM) for ³H-retinol-loaded RBP, BSA and β-lactoglobulin were added to cells to compare retinol uptake activity. The retinol uptake assay was performed identically as described above for ³H-retinol-RBP.

Monitoring vitamin A depletion from holo-RBP after cellular vitamin A uptake: Transfected or untransfected COS-1 cells grown in 6-well dishes were washed with PBS once and incubated with HPLC-purified holo-His-RBP (0.5 uM) diluted in SFM at 37° C. for 8 h. His-RBP remaining in the culture medium after retinol uptake by cells was purified with Ni-NTA resin. His-RBP eluted in 100 mM imidazole from the resin was concentrated with a Microcon centrifugal filter (Millipore). The absorbance spectrum of His-RBP was measured on a Nanoprop spectrophotometer (Nanoprop Technologies Inc.).

A cell-free assay for ³H-retinol uptake: The cell-free assay for ³H-retinol uptake was modified from a published protocol (6). To prepare crude membranes, transfected or untransfected cells were broken up on ice with a mechanical grinder in PBS with protease inhibitors. After centrifugation at 1,000 g for 3 min at 4° C. to remove cell nuclei, cell lysates were diluted in PBS with protease inhibitors and centrifuged at 16,000 g for 30 min at 4° C. to sediment crude membranes. The membrane pellets were resuspended in PBS and briefly sonicated. Each ³H-retinol uptake reaction contained 20 μg of membrane protein. The reactions were carried out in 137 mM NaCl, 2.7 mM KCl, 10 mM NaHPO₄/KH₂PO₄, 0.25 M sucrose, 5 mM DTT, 20 uM BSA, 47 nM ³H-retinol/RBP and protease inhibitors at 37° C. for 10 min. Reactions were stopped by adding a 10× volume of cold PBS. Membranes were pelleted by centrifugation at 16,000 g for 15 min at 4° C. and washed once with cold PBS. Membranes were pelleted and then solubilized in PBS containing 1% Triton X-100, and radioactivity was measured in a scintillation counter. Membrane prepared from STRA6 and LRAT transfected cells takes up about 17% of total radioactivity in 10 minutes (7.5 nM ³H-retinol/RBP in the medium). Radioactivity contributed from nonspecific binding of ³H-retinol/RBP to plastic tubes was determined by performing identical reactions without cellular membranes added.

Antibody production and immunohistochemistry: Polyclonal antibodies against the C-terminal peptide of bovine STRA6 (QAFRKTALPGARPNGAQP (SEQ ID NO:1)) and a peptide within a putative loop region of bovine STRA6 (ALDIGPLTQSPRPSRQAIFC (SEQ ID NO:2)) were developed. Peptides conjugated to KLH were used to immunize rabbits for polyclonal antibody production (Genemed Synthesis, Inc.). Anti-STRA6 antibodies were purified from crude serum using the corresponding peptide conjugated to Affigel (Bio-Rad). All immunohistochemistry experiments shown were done using the purified antibody against the C-terminal peptide. Fresh bovine tissues obtained from a local slaughterhouse were embedded in OCT compound (Tissue-Tek) and frozen in a dry ice/isobutane bath. For immunohistochemistry, cryostat sections of tissues were first washed in PBS⁺ (PBS with 2 mM MgCl₂) to remove OCT and fixed in methanol at 4° C. for 30 min. After rehydration in PBS⁺ and incubation with blocking buffer (5% normal goat serum and 0.3% Triton X-100 in PBS⁺) for 1 h, the sections were incubated with primary antibodies diluted in the blocking buffer at 4° C. overnight. GSL I-isolectin B4 (Vector Laboratories) was added to 10 μg/ml to label endothelial cells. DAPI (Invitrogen) was added to 0.1 μg/ml to label cell nuclei. After washing with PBS⁺ 4 times for 10 min each, the sections were incubated with Alexa Fluor 488 or Alexa Fluor 594 labeled goat anti-rabbit antibody (Invitrogen) diluted in the blocking buffer. After further washes with PBS⁺, the sections were mounted in Vectorshield mounting medium (Vector Laboratories). Fluorescent microscopy was performed using a Nikon Eclipse 80i system. Confocal microscopy was performed using Leica TCS-SP1 system.

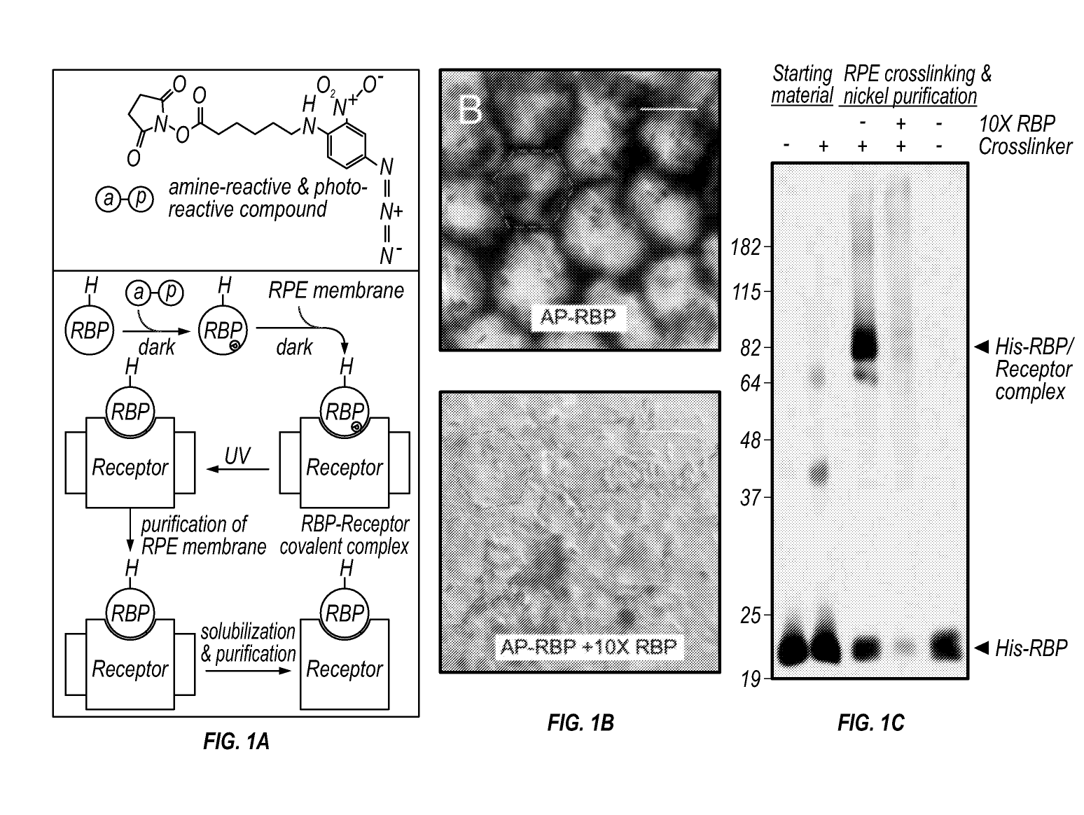

Production of alkaline phosphatase (AP)-RBP fusion protein and binding of AP-RBP to live cells: AP fusion is an effective method to label secreted proteins and study their interactions with cell-surface receptors. In this system, the GPI anchor of human placental AP was removed to make it a secreted protein. AP can be tagged at either the N-terminus or C-terminus of a secreted protein like RBP. N-terminal tagging with AP uses AP's original secretion signal. C-terminal tagging with AP uses the secretion signal of RBP. AP fusion proteins can be harvested from the supernatant of transfected cells. Since this AP is heat resistant, heating is an effective way to eliminate endogenous AP activity. Detection of AP is a simple one-step color reaction, which is ideal for quantitative studies. A structural and functional study of RBP and the 3-D structure of RBP suggest that the RBP N-terminus is not involved in RBP-receptor interaction, while the RBP C-terminus is in close proximity to the regions involved in this interaction. Consistently, N-terminal AP-tagged RBP (AP-RBP) was able to bind to its receptor on RPE cells with a staining pattern expected for basolateral membranes (FIG. 1B). Binding of AP-RBP to live cells was performed similarly as described (10). For AP staining of live cells, fresh RPE cells or COS-1 cells (transfected or untransfected) grown on gelatin-coated coverslips were washed once with PBS and incubated with AP-RBP diluted in SFM for 1 h at room temperature. The cells were then washed twice with PBS⁺ and fixed in fresh 2% formaldehyde in PBS⁺ at room temperature for 10 min. After 3 more PBS washes, the cells were heated in PBS for 1 h at 65° C. The cells were washed once with AP buffer (0.1 M Tris, pH 9.5, 0.1 M NaCl and 50 mM MgCl₂) and then the AP color reactions were performed in AP buffer containing 165 μg/ml of BCIP (Roche) and 330 μg/ml of NBT (Roche) for 1 hour at room temperature. For the liquid AP assay, transfected or untransfected cells grown on 12-well cell culture plates were washed once with PBS and incubated with AP-RBP diluted in SFM at 37° C. for 1 h. The reaction was stopped by washing cells twice with PBS containing 1 mM CaCl₂. The cells were then lysed in 200 μl of cold PBS containing 1% Triton X-100 and protease inhibitors per well. The cell lysates were centrifuged at 3,000 g at 4° C. for 5 min. The supernatants were removed and heated at 65° C. for 1 h. Twenty microliters of the heated lysate were mixed with 200 μl of pNPP (Sigma) and incubated at 37° C. for 1 h for the AP color reaction. The reactions were stopped by adding 50 μl 3 M NaOH and were transferred to a 96-well plate for reading in a microplate reader at 405 nm.

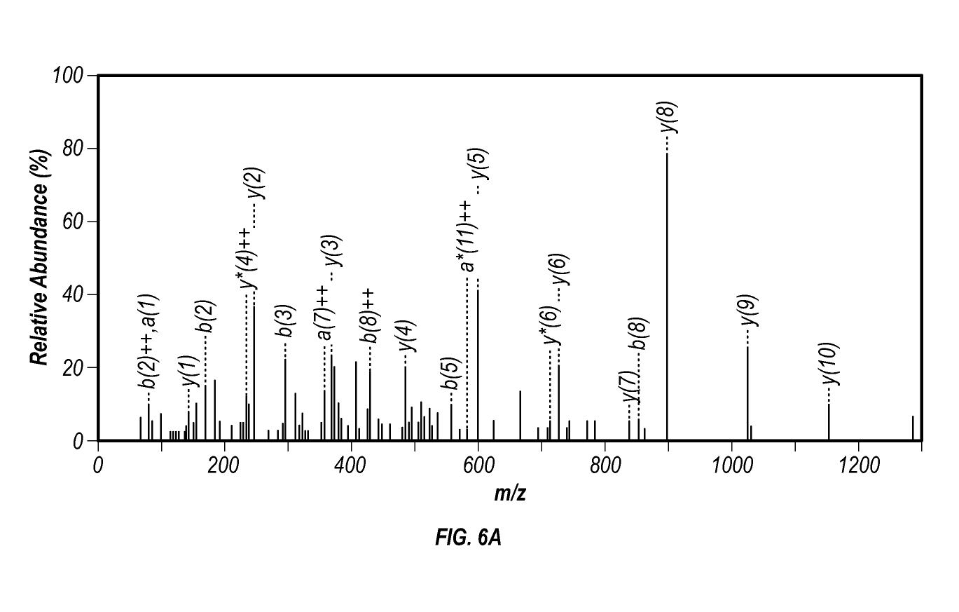

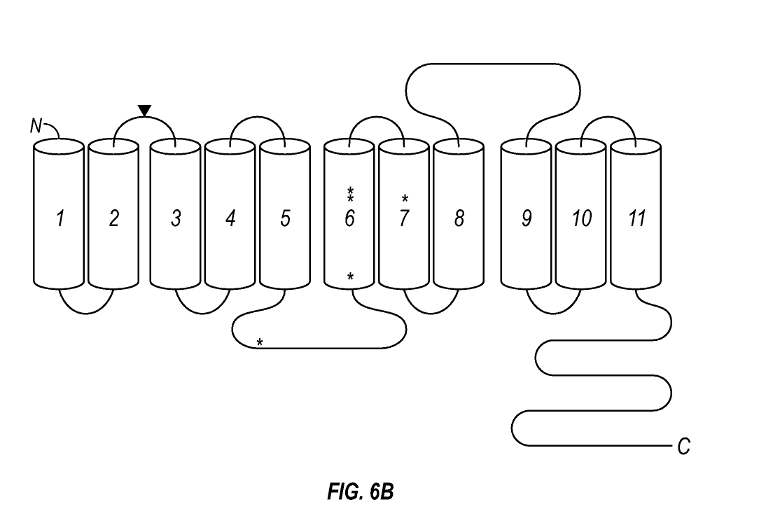

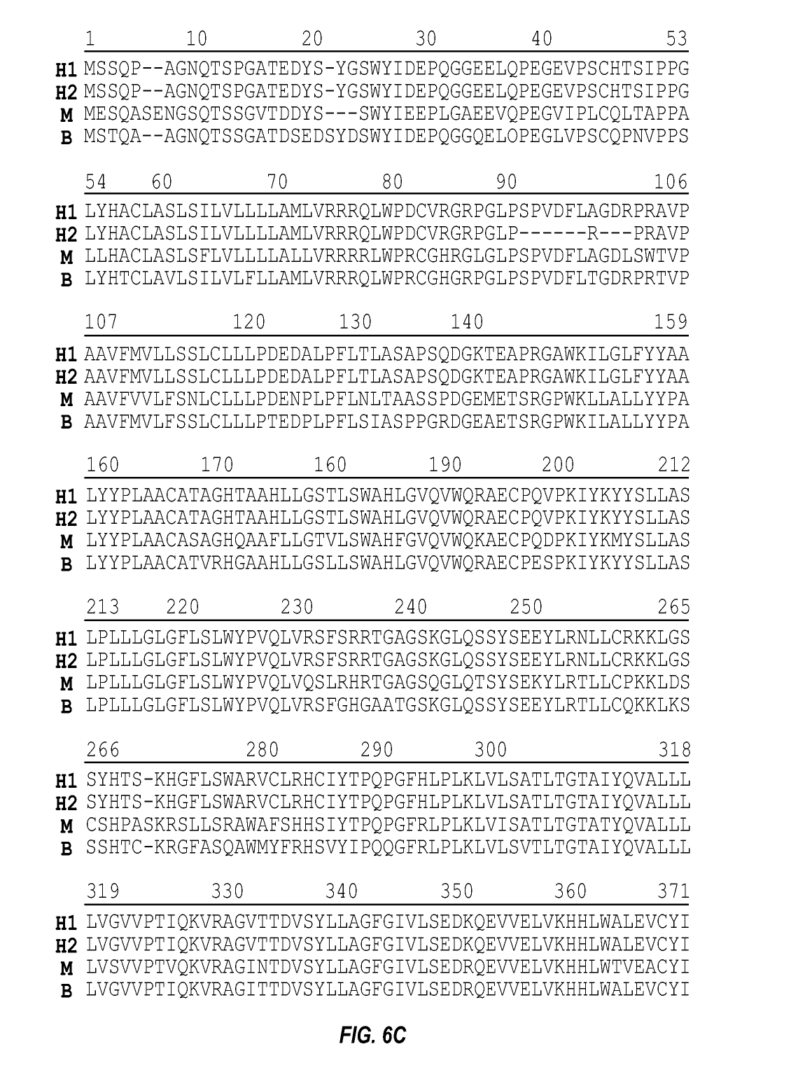

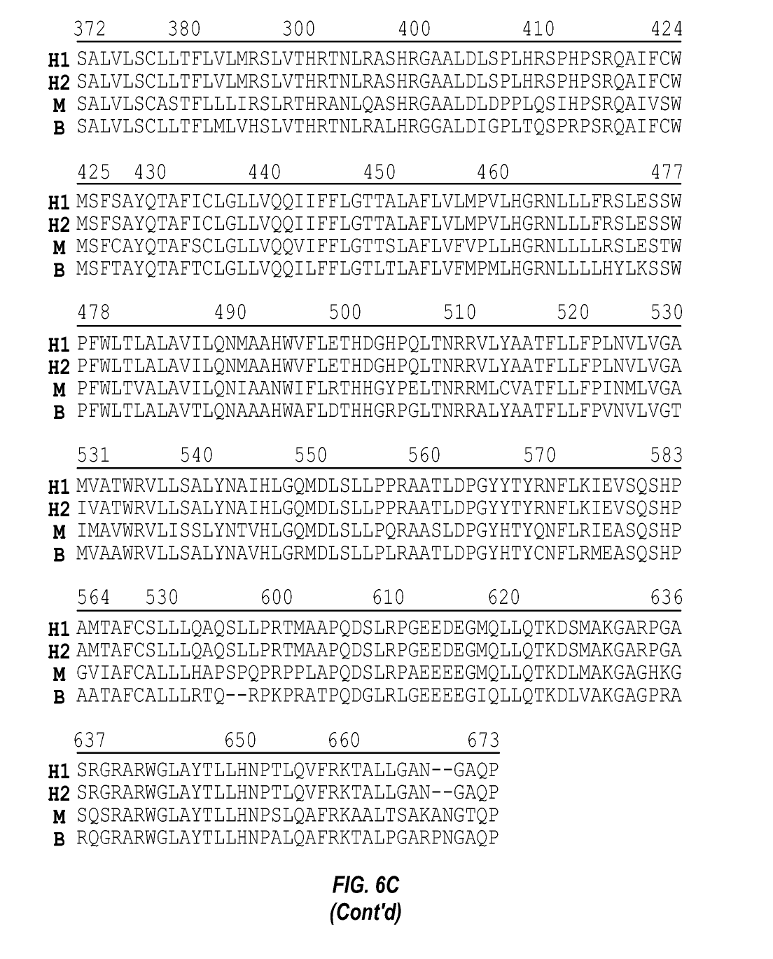

Mutagenesis of bovine STRA6 and characterization of its mutants: Random mutagenesis of bovine STRA6 was performed using the GeneMorph II random mutagenesis system (Stratagene). The protocol was optimized so that each mutant contains about 2 mutations; this is an acceptable rate since not all mutations cause an amino acid change. Vitamin A uptake activity from holo-RBP and AP-RBP binding activity for each mutant was assayed as described above. α-bungarotoxin (BTX) and its high-affinity binding site (BBS) provide an efficient tagging system to study membrane proteins (11). Since the polyclonal antibodies produced do not bind to STRA6 in live cells, a BBS tagging system was used to study the cell surface expression of STRA6 mutants. Insertion of BBS into a loop region of bovine STRA6 (arrowhead in FIG. 6B) confers cell-surface binding to BTX for live cells transfected with this STRA6-BBS construct. This suggests that this loop region of STRA6 is located extracellularly. Mutants were produced in this STRA6-BBS background and determined their cell surface expression by binding to Alexa Fluor 594-conjugated BTX (Invitrogen) on live cells.

RNAi transfection and retinoic acid treatment of human WiDr colon adenocarcinoma cells: WiDr cells (American Type Culture Collection) were grown in Earle's balanced salt media with 10% FBS at 37° C. in a 95% air/5% CO₂ humidified incubator. Human STRA6 RNAi oligo sequences were 5′-UGAAGCUCAUCCAACAGAAUAUGGC-3′ (S-1) (SEQ ID NO:3), 5′-UAGAUGCAGUGUCUCAAGCAGACGC-3′ (S-2) (SEQ ID NO:4); and 5′-UGAGUAAGCAGGACAAGACCAAGGC-3′ (S-3) (SEQ ID NO:5). Stealth™ negative control RNAi with medium GC (Invitrogen) was used for a negative control. WiDr cells were transfected with 100 nM RNAi oligos using DharmaFect1 (Dharmacon) according to the manufacture's protocol. WiDr Cells were split one day before RNAi transfection and cells were incubated with RNAi oligos for 65 hrs after transfection. Retinoic acid treatment was applied for 55 hours. RNA was isolated from WiDr cells using the RNeasy kit (Qiagen). Reverse transcription was performed with Invitrogen's SuperScriptIII First-Strand Synthesis System. Semi-Quantitative PCR was carried out for 24 cycles at 95° C. for 30 sec (denaturation), 63° C. for 30 sec (annealing), and 72° C. for 30 min (extension). cDNA synthesized from 40 ng of total RNA was used for each reaction. Primers used for PCR to detect human STRA6 were 5′-CTGGAGTCCTCGTGGCCCTTCTGGCTGAC-3′ (human STRA6 forward primer (SEQ ID NO:6)), 5′-TCGGTACGTGTAGTAGCCGGGGTCGAGAGTG-3′ (human STRA6 reverse primer (SEQ ID NO:7)).

Primary culture and RNAi transfection of bovine RPE cells: Primary cultures of RPE cells were derived from adult bovine eyes obtained from a local slaughterhouse. After the removal of vitreous and retina, the posterior half of the eye was incubated in 2% Dispase solution (Boehringer Mannheim) for 2 h. Sheets of RPE cells were then collected and washed with Hanks' Balanced salt solution (Irvine Scientific). The RPE cells were exposed to 0.05% Trypsin and 0.02% EDTA solution for 5 minutes at room temperature. The RPE cells were suspended in CEM replacement medium containing 10% calf serum and 1% penicillin-streptomycin as described previously (12), and the cells were then seeded at a density of 3×10⁵ cells/cm² onto 12 mm millicell-PCF culture wells with a pore size of 0.45 um (Millipore Corp.). The RPE cells were maintained at 37° C. in a 5% CO₂ incubator for 10 days before RNAi transfection. For RNAi transfection, bovine RPE cells were washed once with PBS and incubated in fresh DMEM/10% FBS media. Cells were transfected with 100 nM RNAi oligos (Stealth RNAi, Invitrogen) using DharmaFect1 according to manufacturer's protocol and incubated at 37° C. for 72 h. Cells were then transfected under the same conditions and incubated for another 72 h. RNAi oligo sequences used for this assay were 5′-CCGUCUACAUUCCACAACAAGGAUU-3′ (S-4) (SEQ ID NO:8), 5′-GGCUACCACACAUACUGCAACUUCU-3′ (S-5) (SEQ ID NO:9) and 5′-GAGGAUUCCUAUGACAGCUGGUACA-3′ (S-6) (SEQ ID NO:10). Semi-quantitative PCR was carried out for 26 cycles at 95° C. for 30 sec (denaturation), 60° C. for 30 sec (annealing), and 72° C. for 30 min (extension). cDNA synthesized from 32 ng of total RNA was used for each reaction. Primers used for PCR reaction to detect bovine STRA6 are 5′-CCAAAGGCTCAGGAATCCGAGG-3′ (forward primer (SEQ ID NO:11)), 5′-ATGTCCACCCAGGCGGCAGGGAACC-3′ (reverse primer (SEQ ID NO:12)).

Identification of the RBP receptor as STRA6. Potential obstacles to purifying the RBP receptor include the fragility of the receptor protein and the transient nature of binding. In one aspect, the disclosure provides methods for stabilizing the RBP-receptor interaction, and this method permitted high affinity purification of the RBP-receptor complex. The strategy combined a bifunctional crosslinker with an amine reactive group and a photoreactive group and the 6× histidine tag (His tag)-nickel system (FIG. 1A). RPE cells were used as the starting material because they are known to express the RBP receptor at high levels (13). Since N-terminal alkaline phosphatase-tagged RBP was able to specifically bind to its receptor on RPE cells with a staining pattern expected for basolateral membranes (FIG. 1B), His-tagged RBP (His-RBP) were generated by tagging at the N-terminus of RBP. After binding purified His-RBP protein conjugated with the crosslinker to RPE membranes, UV crosslinking, membrane solubilization and nickel resin purification, a covalent protein complex of about 80 kD containing RBP and its putative receptor were observed (FIG. 1C). Mass spectrometry analysis revealed that the RBP binding protein in the complex was STRA6, a protein that has 11 putative transmembrane domains but has no homology to any protein with known function (FIG. 6). STRA6 is a retinoic acid induced gene in P19 embryonic carcinoma cells.

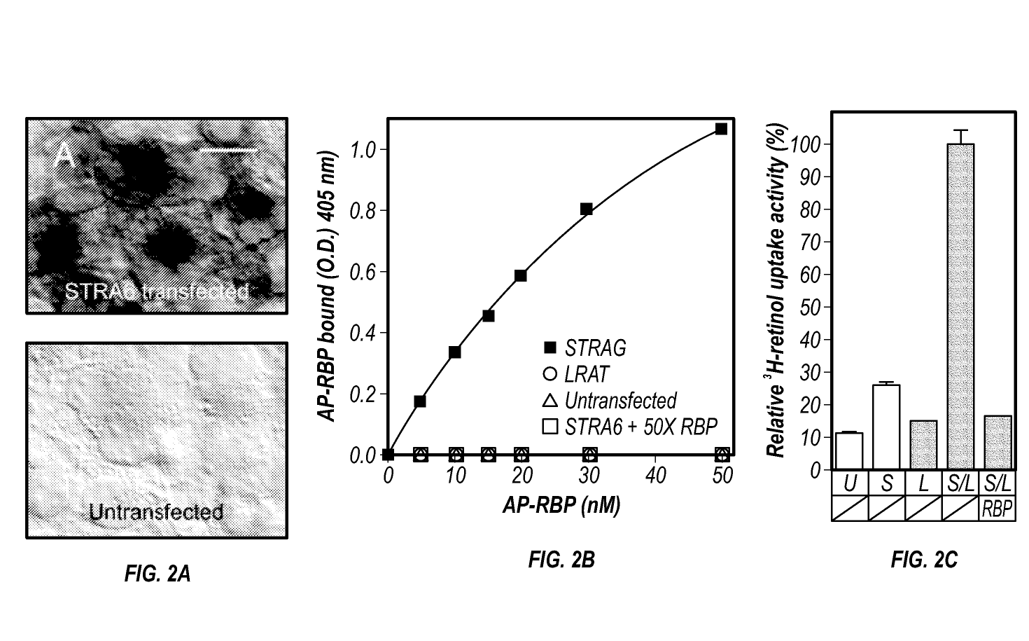

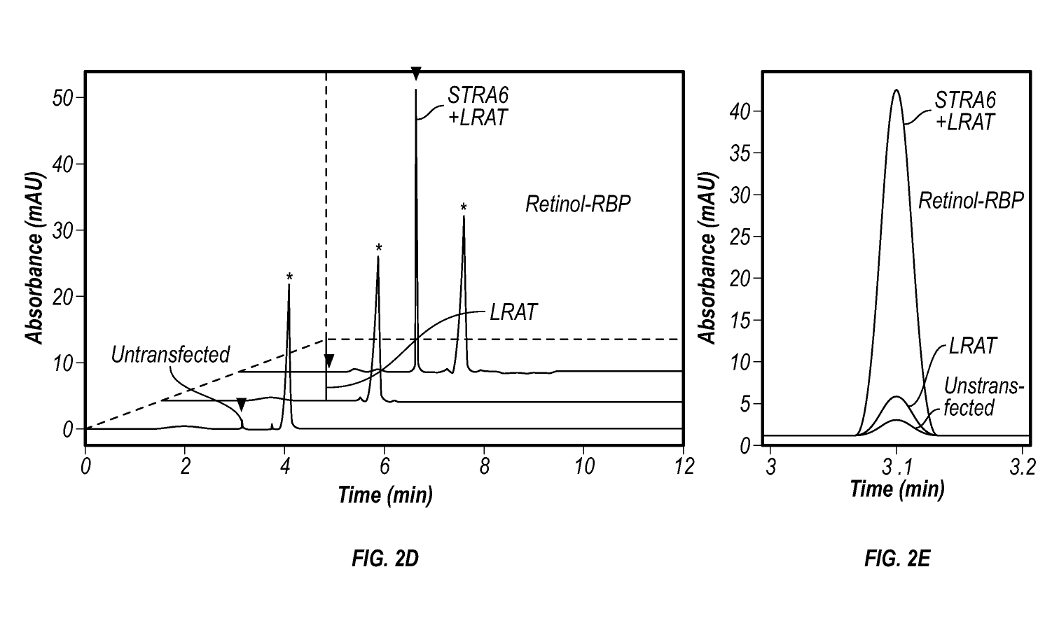

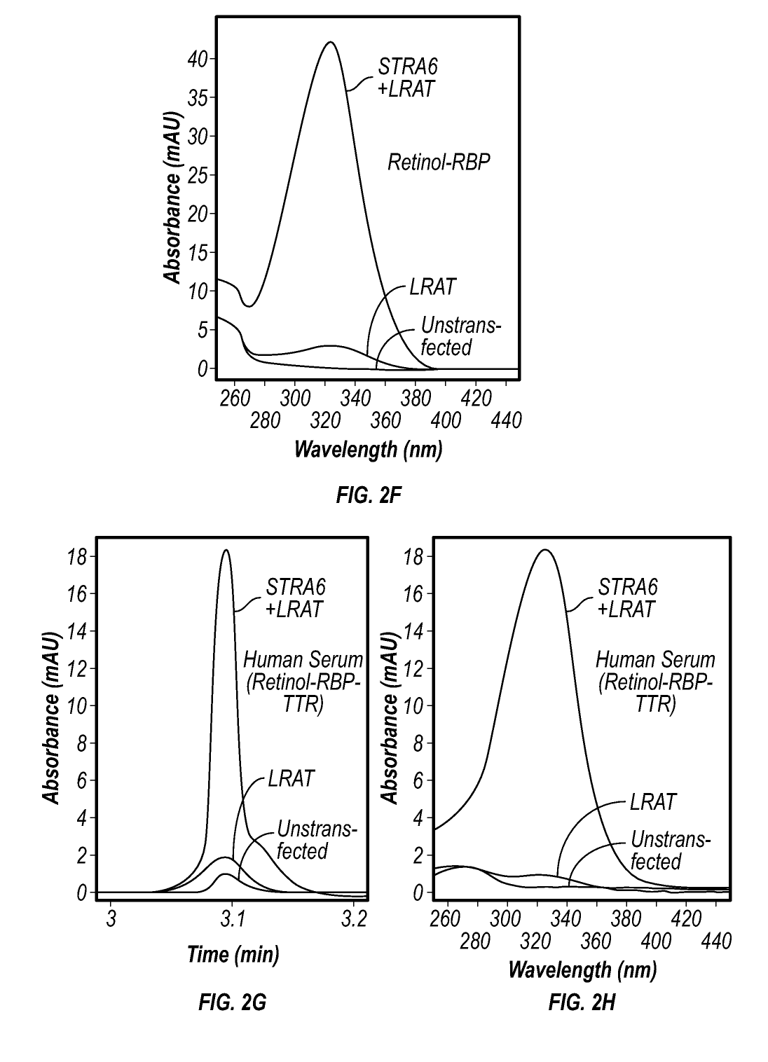

STRA6 mediates RBP binding and vitamin A uptake from holo-RBP. To test whether STRA6 could confer RBP binding to transfected cells, bovine STRA6 cDNA was transfected into COS-1 cells. Bovine STRA6-transfected but not untransfected cells bound to AP-RBP with high affinity (Kd=59 nM) (FIGS. 2A and 2B). An excess of unlabeled RBP blocked this binding activity (FIG. 2B). To test whether STRA6 could enhance cellular vitamin A uptake from holo-RBP, both ³H-retinol-based assays (FIG. 2C) and HPLC-based assays (FIG. 2D-2H) were performed. STRA6 expression dramatically increased cellular vitamin A uptake from holo-RBP. Vitamin A is preferentially stored as retinyl esters after uptake from holo-RBP. Lecithin retinol acyltransferase (LRAT) is the enzyme that converts retinol into retinyl ester and plays an important role in vitamin A storage. LRAT expression was included in most vitamin A uptake assays. Using an assay based on ³H-retinol-RBP, STRA6 activity was most evident in the presence of LRAT (FIG. 2C) because LRAT makes it possible to store vitamin A at high concentration in the form of retinyl esters, the more stable derivative of vitamin A. In these assays, virtually all specific vitamin A uptake activity, which was sensitive to blocking by an excess of unlabeled RBP, was due to STRA6. Vitamin A uptake by COS-1 cells transfected with STRA6 and LRAT is highly efficient. When the concentration of ³H-retinol-RBP is 3 nM in the medium, about 25% of the total radioactivity was taken up by cells in 1 hour, and about 50% of the total radioactivity was taken up by cells in 2 hours. The amount of retinol taken up by cells in 1 hour is about 4.6 times the moles of RBP bound to the cell surface at steady state. HPLC was also used to analyze vitamin A uptake mediated by STRA6 (FIG. 2D-2H). In this assay, untransfected COS-1 cells accumulated little retinyl ester. COS-1 cells transfected with LRAT alone did accumulate a small amount of retinyl esters, but cells cotransfected with STRA6 and LRAT showed a 15-fold higher level of retinyl ester accumulation than cells transfected with LRAT alone (FIG. 2D-2F). At the saturating retinol-RBP concentration (0.44 μM) used for the HPLC assays, the amount of retinol taken up by cells in 1 hour is about 14 times the moles of RBP bound to the cell surface at steady state. Holo-RBP in the blood is in complex with the thyroxine binding protein, transthyretin (TTR). Although TTR blocks the vitamin A exit site in the holo-RBP-TTR complex, the fact that tissues take up vitamin A from holo-RBP-TTR in the blood in vivo suggests that TTR cannot completely inhibit tissue vitamin A uptake from holo-RBP. When vitamin A uptake was assayed using human serum as the source of the holo-RBP-TTR complex, STRA6 could still efficiently take up vitamin A from this complex (FIG. 2G-2H).

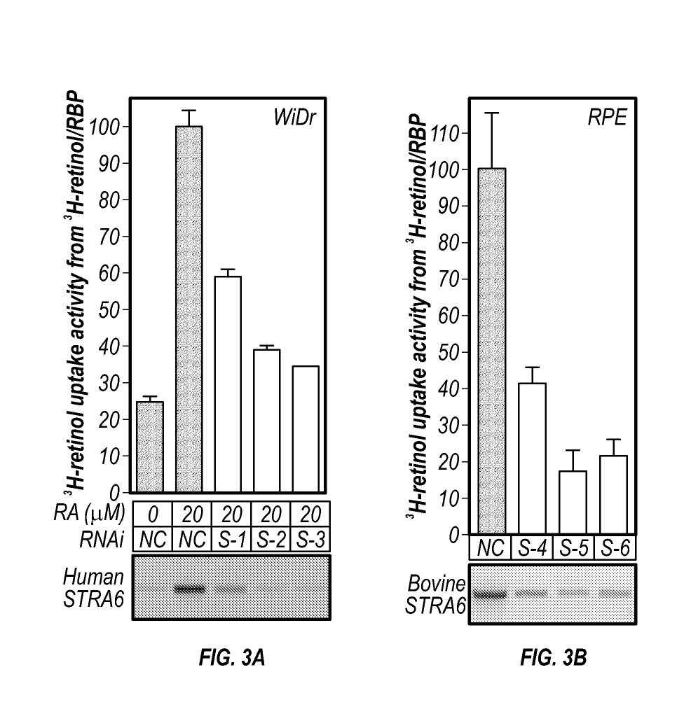

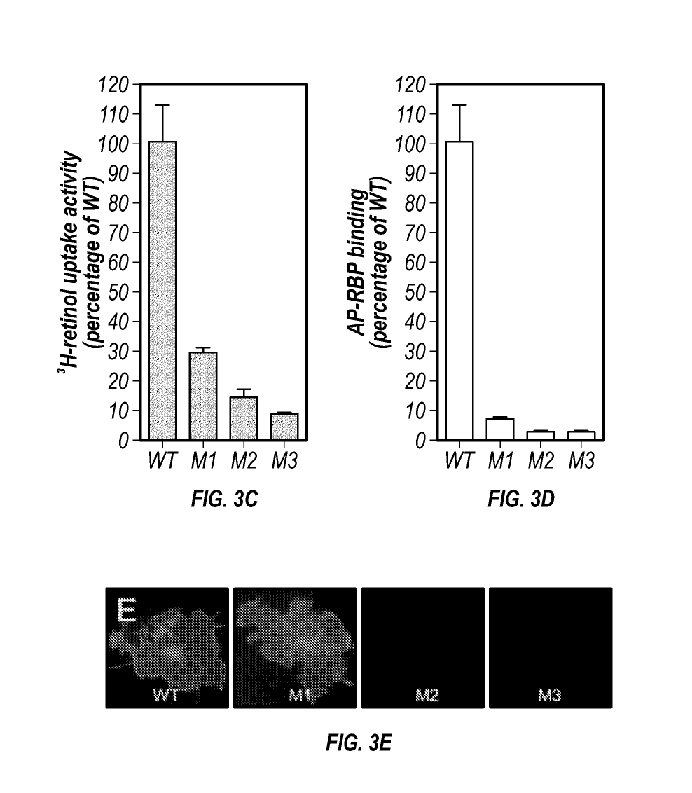

Because retinoic acid upregulates STRA6 in certain cancer cell lines such as WiDr human colon adenocarcinoma cells, WiDr cells were used as an independent model to study vitamin A uptake mediated by STRA6. STRA6 expression is increased in WiDr cells by retinoic acid treatment and decreased its expression by RNAi. Consistent with STRA6's function in mediating vitamin A uptake from holo-RBP, its increased expression by retinoic acid treatment enhanced vitamin A uptake activity while its decreased expression by specific RNAi knockdown suppressed vitamin A uptake activity (FIG. 3A). In addition, RNAi knockdown experiments were performed on primary bovine RPE cultures. Again a decrease in STRA6 expression suppressed RPE's vitamin A uptake activity from holo-RBP (FIG. 3B).

Because STRA6 is not homologous to any protein of known function and has never been characterized at the structural level, an unbiased strategy was used to test the effects of mutations on STRA6 function. 50 random missense mutants of STRA6 were generated and characterized; 3 of them resulted in significant loss of vitamin A uptake activity (FIG. 3C-3E). The locations of these mutations in the putative transmembrane topology model of STRA6 are shown in FIG. 6B. Mutants 2 and 3 (M2 and M3) were not expressed at the cell surface, which may be sufficient to account for the dramatic loss of RBP binding and vitamin A uptake activity from holo-RBP. Mutant 1 (M1) was normally expressed at the cell surface, but had significantly reduced RBP binding and vitamin A uptake activity. The loss of RBP binding for M1 may account for its lower vitamin A uptake activity.

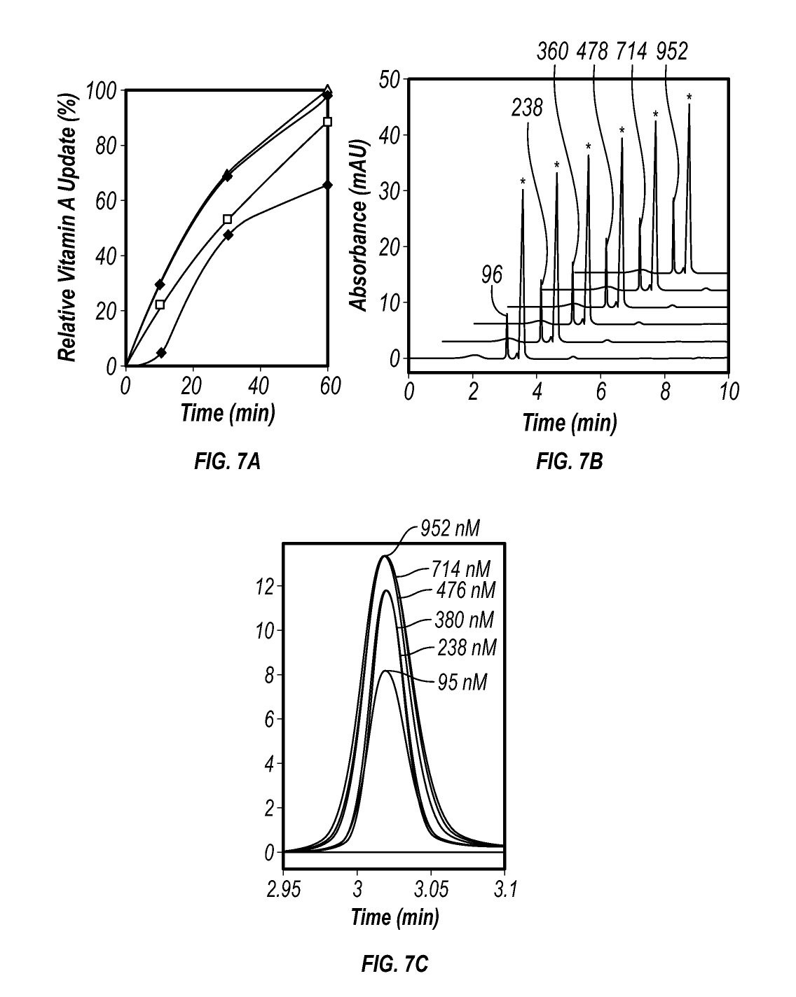

Characterization of STRA6-mediated vitamin A uptake from holo-RBP. STRA6-mediated vitamin A uptake is specific to RBP. STRA6 could not enhance cellular uptake of vitamin A when vitamin A was bound to BSA or β-lactoglobulin (FIG. 4A). STRA6-mediated vitamin A uptake was saturable with regard to retinol-RBP concentration (FIG. 4B and FIG. 7). A variety of studies from the past three decades indicate that endocytosis is not required for RBP receptor-mediated vitamin A uptake from holo-RBP. In agreement with these studies, metabolic inhibitors that effectively inhibited endocytosis did not inhibit STRA6-mediated vitamin A uptake by STRA6-transfected COS-1 cells or retinoic acid-stimulated WiDr cells (FIGS. 4C and 4D). Membranes prepared from transfected cells have vitamin A uptake activity similar to that of live cells (FIG. 4E). The robust activity of this cell-free system also argues against an endocytosis-based mechanism. An assay was developed that monitored both RBP and vitamin A that remains bound to RBP in the culture medium after cellular vitamin A uptake. RBP in the cell culture medium was purified following cellular uptake of vitamin A and the absorption spectrum of the purified RBP was measured (FIG. 4F). A preferential decrease in the vitamin A peak for RBP taken from the medium of STRA6 and LRAT-transfected cells again argued against an endocytosis-based mechanism.

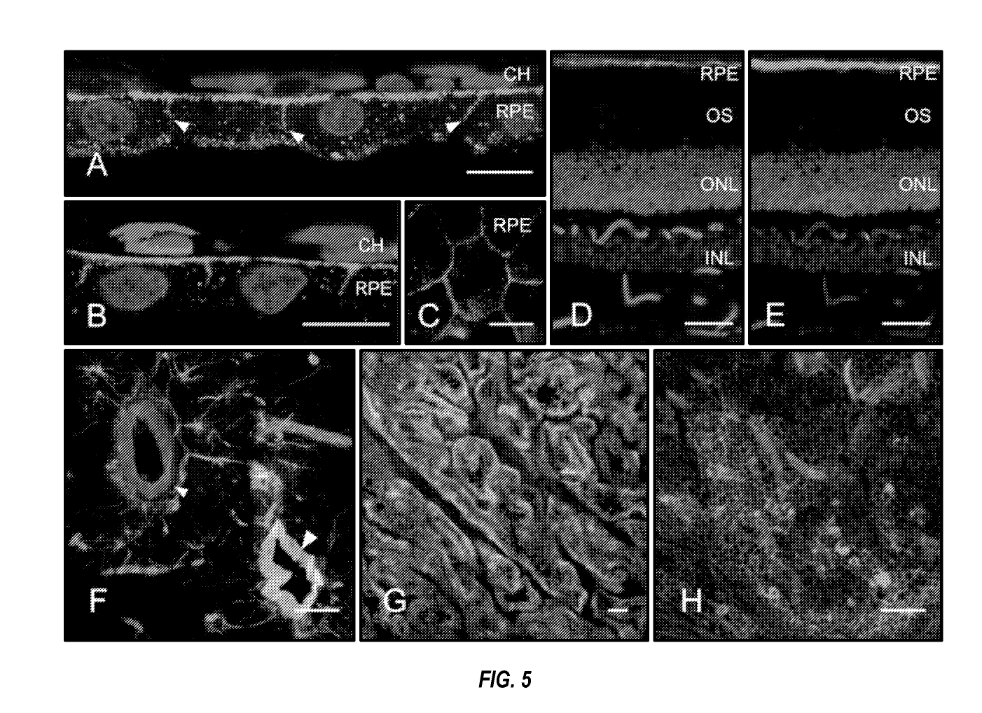

Localization of STRA6 is consistent with its function as the RBP receptor. STRA6 is expressed during embryonic development, in the brain, spleen, kidney, female genital tract and testis (and at lower levels in heart and lung). Furthermore, STRA6 is highly enriched in the RPE in the adult eye. To further study the location of STRA6 in adult organs, polyclonal antibodies were produced against bovine STRA6 and immunohistochemistry was performed on several adult organs. In the RPE, STRA6 is localized to the basolateral membrane (FIG. 5A-5C), a location consistent with its role in interacting with RBP in the choriocapillaris blood. In contrast to its absence in the endothelial cells of the choriocapillaris (FIGS. 5A and 5B), STRA6 was expressed in retinal blood vessels, another location of the blood-retina barrier (FIGS. 5D and 5E). Holo-RBP from retinal blood vessels is a potential source of vitamin A for Müller cells in the retina. In addition to blood-brain barriers, STRA6 expression was identified in astrocyte perivascular endfeet that surround blood vessels negative for STRA6 (FIG. 5F). In accord with NCBI's EST tissue expression profile for STRA6, STRA6 expression was observed in the placenta (FIG. 5G), and the spleen (FIG. 5H).

A number of embodiments have been described. Nevertheless, it will be understood that various modifications may be made without departing from the spirit and scope of the description. Accordingly, other embodiments are within the scope of the following claims.

What is claimed is:

1. A method of diagnosing a condition associated with abnormal uptake of a retinoid comprising determining the amount of STRA6 expressed by a suspect cell and comparing the amount of expressed STRA6 in the suspect cell with the amount of expressed STRA6 in a normal cell.

2. The method of claim 1 wherein the amount of expressed STRA6 is determined using an antibody which specifically binds STRA6.

3. The method of claim 1 wherein the amount of expressed STRA6 is determined using PCR with primers specific for STRA6.

4. The method of claim 1 wherein the condition associated with abnormal uptake of a retinoid is a condition in which an insufficient amount of retinoid is taken up by the suspect cell.

5. The method of claim 1 wherein the condition associated with abnormal uptake of a retinoid is a condition in which an excessive amount of retinoid is taken up by the suspect cell.

6. The method of claim 1 wherein the condition associated with abnormal uptake of a retinoid is a visual disorder, neurological disorder, cancer, diabetes, skin disease, immune disorder, or lung disease.

7. A method of treating a condition associated with excessive uptake of a retinoid comprising administering to a subject in need thereof an amount of a drug effective for lowering expression or activity of STRA6 in cells with excessive retinoid uptake.

8. The method of claim 7 wherein siRNA for STRA6 is administered to cells with excessive retinoid uptake.

9. The method of claim 7 wherein the condition associated with excessive uptake of a retinoid is a visual disorder, neurological disorder, cancer, diabetes, skin disease, immune disorder, or lung disease.

10. The method of claim 9 wherein the condition treated is a visual disorder.

11. The method of claim 10 wherein the visual disorder is one in which there is excessive uptake of a retinoid in a retinal pigmented epithelial cell.

12. The method of claim 10 wherein the visual disorder is diabetic retinopathy.

13. The method of claim 10 wherein the visual disorder is age-related macular degeneration.

14. The method of claim 13 wherein administration of the drug reduces uptake of a retinoid by a retinal pigmented epithelial cell.

15. A method of treating a condition associated with insufficient uptake of a retinoid comprising administering to a subject in need thereof an amount of a drug effective for increasing expression or activity of STRA6 in cells with insufficient retinoid uptake.

16. The method of claim 15 wherein exogenous nucleic acid encoding STRA6 is transfected into cells with insufficient retinoid uptake.

17. The method of claim 15 wherein the condition associated with insufficient uptake of a retinoid is a visual disorder, neurological disorder, cancer, diabetes, skin disease, immune disorder, or lung disease.

18. The method of claim 17 wherein the condition treated is a visual disorder.

19. The method of claim 18 wherein the visual disorder is one in which there is insufficient uptake of a retinoid in a retinal pigmented epithelial cell.

20. The method of claim 18 wherein the visual disorder is diabetic retinopathy.

21. The method of claim 18 wherein the visual disorder is age-related macular degeneration.

22. A method of screening for a compound which alters the uptake of a retinoid by a cell, the method comprising exposing the cell to the compound and determining if the expression or activity of STRA6 is altered.

23. The method of claim 22 wherein exposing the cell to the compound takes place in vitro.

24. The method of claim 22 wherein exposing the cell to the compound takes place in vivo.

25. A transfected cell, the cell comprising exogenous DNA encoding STRA6.

26. A transgenic animal, the animal comprising cells transfected with DNA encoding STRA6.


SEQUENCE LISTING (17 sequences)

SEQ ID NO: 1 - protein, 18 aa, Artificial Sequence
  Peptide fragment from bovine STRA6
QAFRKTALPGARPNGAQP

SEQ ID NO: 2 - protein, 20 aa, Artificial Sequence
  Peptide fragment from bovine STRA6
ALDIGPLTQSPRPSRQAIFC

SEQ ID NO: 3 - RNA, 25 nt, Artificial Sequence
  RNAi sequence of human STRA6
ugaagcucauccaacagaauauggc

SEQ ID NO: 4 - RNA, 25 nt, Artificial Sequence
  RNAi sequence of human STRA6
uagaugcagugucucaagcagacgc

SEQ ID NO: 5 - RNA, 25 nt, Artificial Sequence
  RNAi sequence for human STRA6
ugaguaagcaggacaagaccaaggc

SEQ ID NO: 6 - DNA, 29 bp, Artificial Sequence
  Human STRA6 forward primer
ctggagtcctcgtggcccttctggctgac

SEQ ID NO: 7 - DNA, 31 bp, Artificial Sequence
  human STRA6 reverse primer
tcggtacgtgtagtagccggggtcgagagtg

SEQ ID NO: 8 - RNA, 25 nt, Artificial Sequence
  RNAi oligonucleotide
ccgucuacauuccacaacaaggauu

SEQ ID NO: 9 - RNA, 25 nt, Artificial Sequence
  RNAi oligonucleotide
ggcuaccacacauacugcaacuucu

SEQ ID NO: 10 - RNA, 25 nt, Artificial Sequence
  RNAi oligonucleotide
gaggauuccuaugacagcugguaca

SEQ ID NO: 11 - DNA, 22 bp, Artificial Sequence
  bovine STRA6 forward primer
ccaaaggctcaggaatccgagg

SEQ ID NO: 12 - DNA, 25 bp, Artificial Sequence
  bovine STRA6 reverse primer
atgtccacccaggcggcagggaacc

SEQ ID NO: 13 - protein, 14 aa, Artificial Sequence
  Peptide fragment from bovine STRA6
LGEEEEGIQLLQTK

SEQ ID NO: 14 - protein, 667 aa, Homo sapiens
MSSQPAGNQTSPGATEDYSYGSWYIDEPQGGEELQPEGEVPSCHTSIPPGLYHACLASLSILVLLLLAMLVRRRQLWPDCVRGRPGLPSPVDFLAGDRPRAVPAAVFMVLLSSLCLLLPDEDALPFLTLASAPSQDGKTEAPRGAWKILGLFYYAALYYPLAACATAGHTAAHLLGSTLSWAHLGVQVWQRAECPQVPKIYKYYSLLASLPLLLGLGFLSLWYPVQLVRSFSRRTGAGSKGLQSSYSEEYLRNLLCRKKLGSSYHTSKHGFLSWARVCLRHCIYTPQPGFHLPLKLVLSATLTGTAIYQVALLLLVGVVPTIQKVRAGVTTDVSYLLAGFGIVLSEDKQEVVELVKHHLWALEVCYISALVLSCLLTFLVLMRSLVTHRTNLRALHRGAALDLSPLHRSPHPSRQAIFCWMSFSAYQTAFICLGLLVQQIIFFLGTTALAFLVLMPVLHGRNLLLFRSLESSWPFWLTLALAVILQNMAAHWVFLETHDGHPQLTNRRVLYAATFLLFPLNVLVGAMVATWRVLLSALYNAIHLGQMDLSLLPPRAATLDPGYYTYRNFLKIEVSQSHPAMTAFCSLLLQAQSLLPRTMAAPQDSLRPGEEDEGMQLLQTKDSMAKGARPGASRGRARWGLAYTLLHNPTLQVFRKTALLGANGAQP

SEQ ID NO: 15 - protein, 658 aa, Homo sapiens [sequence omitted]

SEQ ID NO: 16 - protein, 668 aa, Bos taurus
MSTQAAGNQTSSGATDSEDSYDSWYIDEPQGGQELQPEGLVPSCQPNVPPSLYHTCLAVLSILVLFLLAMLVRRRQLWPRCGHGRPGLPSPVDFLTGDRPRTVPAAVFMVLFSSLCLLLPTEDPLPFLSLASPPGRDGEAETSRGPWKILALLYYPALYYPLAACATVRHGAAHLLGSLLSWAHLGVQVWQRAECPESPKIYKYYSLLASLPLLLGLGFLSLWYPVQLVRSFGHGAATGSKGLQSSYSEEYLRTLLCQKKLKSSSHTCKRGFASQAWMYFRHSVYIPQQGFRLPLKLVLSVTLTGTAIYQVALLLLVGVVPTIQKVRAGITTDVSYLLAGFGIVLSEDRQEVVELVKHHLWALEVCYISALVLSCLLTFLMLVHSLVTHRTNLRALHRGGALDIGPLTQSPRPSRQAIFCWMSFTAYQTAFTCLGLLVQQILFFLGTLTLAFLVFMPMLHGRNLLLLHYLKSSWPFWLTLALAVTLQNAAAHWAFLDTHHGRPGLTNRRALYAATFLLFPVNVLVGTMVAAWRVLLSALYNAVHLGRMDLSLLPLRAATLDPGYHTYCNFLRMEASQSHPAATAFCALLLRTQRPKPRATPQDGLRLGEEEEGIQLLQTKDLVAKGAGPRARQGRARWGLAYTLLHNPALQAFRKTALPGARPNGAQP

SEQ ID NO: 17 - protein, 670 aa, Mus musculus
MESQASENGSQTSSGVTDDYSSWYIEEPLGAEEVQPEGVIPLCQLTAPPALLHACLASLSFLVLLLLALLVRRRRLWPRCGHRGLGLPSPVDFLAGDLSWTVPAAVFVVLFSNLCLLLPDENPLPFLNLTAASSPDGEMETSRGPWKLLALLYYPALYYPLAACASAGHQAAFLLGTVLSWAHFGVQVWQKAECPQDPKIYKHYSLLASLPLLLGLGFLSLWYPVQLVQSLRHRTGAGSQGLQTSYSEKYLRTLLCPKKLDSCSHPASKRSLLSRAWAFSHHSIYTPQPGFRLPLKLVISATLTGTATYQVALLLLVSVVPTVQKVRAGINTDVSYLLAGFGIVLSEDRQEVVELVKHHLWTVEACYISALVLSCASTFLLLIRSLRTHRANLQALHRGAALDLDPPLQSIHPSRQAIVSWMSFCAYQTAFSCLGLLVQQVIFFLGTTSLAFLVFVPLLHGRNLLLLRSLESTWPFWLTVALAVILQNIAANWIFLRTHHGYPELTNRRMLCVATFLLFPINMLVGAIMAVWRVLISSLYNTVHLGQMDLSLLPQRAASLDPGYHTYQNFLRIEASQSHPGVIAFCALLLHAPSPQPRPPLAPQDSLRPAEEEEGMQLLQTKDLMAKGAGHKGSQSRARWGLAYTLLHNPSLQAFRKAALTSAKANGTQP